(19) United States
(12) Patent Application Publication
(10) Pub. No.: US 2022/0313442 A1
(43) Pub. Date: Oct. 6, 2022

(54) OSTEOGENIC AND ANGIOGENIC IMPLANT MATERIAL

(71) Applicant: Anthony Ruggiero, Dana Point, CA (US)

(72) Inventor: Anthony Ruggiero, Dana Point, CA (US)

(21) Appl. No.: 17/218,755

(22) Filed: Mar. 31, 2021

(51) Int. Cl. A61F 2/30; B33Y 10/00; B33Y 80/00; B33Y 40/10; B33Y 40/20; B29C 64/314; B29C 71/00

(52) U.S. Cl. CPC A61F 2/30771; A61F 2/30942; B33Y 10/00; B33Y 80/00; B33Y 40/10; B33Y 40/20; B29C 64/314; B29C 71/0009; A61F 2002/3092; A61F 2002/30242; A61F 2002/30112; A61F 2002/30985; A61F 2002/30823; A61F 2002/30263; A61F 2002/30273; A61F 2002/30677; A61F 2310/00005; B29K 2509/02

(57) ABSTRACT

A method of manufacturing an implant, including mixing a first quantity of biocompatible polymer particles, a second quantity of bioactive ceramic particles, and a third quantity of fugitive material particles to define an admixture, forming the admixture to define a composite body having an inferior portion, a superior portion and a central portion disposed between the inferior and superior portions, heating the admixture to fuse the first quantity of bioactive polymer particles to define a composite implant body, and infiltrating the composite implant body with a solvent to remove fugitive material particles to yield a network of interconnected pores and to define a porous implant body. The fugitive material particles are hollow spheres partially filled with a material selected from the group comprising air, bioactive agents, biological growth enhancers, drugs, and biocompatible polymer material, combinations thereof.

### TECHNICAL FIELD

The novel technology relates generally to orthopedic medicine, and, more particularly, to implantable bone support devices.

### BACKGROUND

Novel composite materials have been developed that represent a significant evolution over existing implant materials, such as those incorporating polyetheretherketone (PEEK), which can impart strength to physiologic environments requiring load bearing support, such as, for example, interbody spine implants. One advantage of PEEK and like materials is that they provide good osteogenic and angiogenic properties which both reduce fibrous tissue encapsulation and render radiopacity more appropriate for determination of the orientation of the implant.

Incorporation of bioactive ceramic fillers and/or minerals into the base polymer structure to produce a tailored composite is a method that yields consistent mechanical property retention, while simultaneously increasing the ability of the material to bond to bone after implantation. This bonding to bone at key interfaces leads to improved fusion stability, pain relief and accelerated healing. The addition of specific bioceramic materials, such as borate glass, ceramics and glass-ceramics, having a variety of compositional flexibilities but essentially consisting of any bioactive class of ceramics, further enhances these combined properties.

Use of precise placement of these bioactive components into the opposing superior and inferior surfaces of an implant, as well as the interior column, would allow for accelerated load transduction as well as clear orientation of the implant radiographically. The time related healing and bonding of bone tissue to these implants will lend itself to sustained microenvironment conducive to bone apposition and supportive angiogenesis, where vasculature is challenged, but required for consistent bone formation.

Previous attempts to augment or create a bonding interface have included addition of titanium metal, porous interfaces, and hydroxyapatite ceramics (HA). While useful, these improvements have been limited. Further, tissue bonding and regrowth remains a slow and painful process. There remains a need for implant materials that give rise to improved bonding speed and strength as well as improved and more complete osteogenesis/angiogenesis. The present novel technology addresses these needs.

### DESCRIPTION OF THE DRAWINGS

FIGS. 1A-1D are perspective views of a layered composite material body, including the porous and bioactive material configuration of a first embodiment of the present novel technology.

FIGS. 2A-2B are perspective cutaway views of implants according to the present novel technology.

FIG. 3 is a perspective cutaway view of the layered composite body of FIG. 1A-1D according to the present novel technology.

FIGS. 4A-4F are elevation sectional views of multilayer/multicomponent composite beads for use in a polymer matrix according to the present novel technology.

FIGS. 5A-5D are elevation views of various porous polymer matrices having polymer beads partially filling come pores according to the present novel technology.

FIGS. 6A-6B are perspective views of layered porous composite implants according to the present novel technology.

FIGS. 7A-7B are plan cutaway views of composite bodies according to the present novel technology.

FIGS. 8A-8C are partial perspective views of contoured implant bodies according to the present novel technology.

FIG. 9A is a perspective view of a porous composite body according to the present novel technology.

FIGS. 9B-9C are perspective views of a interlockingly connectible porous composite body according to the present novel technology.

FIG. 10 is a flow chart representing a method for the preparation of the implant devices of the present novel technology.

### DESCRIPTION OF PREFERRED EMBODIMENTS

For the purposes of promoting an understanding of the principles of the novel technology, reference will now be made to the embodiments illustrated in the drawings and specific language will be used to describe the same. It will nevertheless be understood that no limitation of the scope of the novel technology is thereby intended, such alterations and further modifications in the illustrated device, and such further applications of the principles of the novel technology as illustrated therein being contemplated as would normally occur to one skilled in the art to which the novel technology relates.

### Overview

The present novel technology relates to multicomponent bioactive ceramic (typically bioglass)-polymer matrix composite bodies having integrated and engineered porous architecture such as defined by fugitive and/or bioactive spheres in a structural matrix. The bodies exhibit sufficient strength to bear load in the human skeleton, especially the spine, allowing for bone apposition, integration and incorporation. This approach is tailorable to yield a predetermined PSD as well as to increase porosity (and, accordingly, available surface area) up to 80% or even 90% or more, while maintaining sufficient strength to support and bear load, thus allowing for physiologic integration. Porosity, porosity gradient, and pore size distribution may be engineered and thus predetermined and varied throughout a given body. Integration of the body with the recipient can be with hard or soft tissue, typically bone formation for hard tissue and muscle and/or collagen formation and growth with soft tissue integration. Some examples of hard tissue integration applications of the present novel technology are spine implants, long bone anchors, implants, interference screws (both in hard and soft tissue), tendon augmentation, bone-tendon interface augmentation or coupling, replacement of soft tissue defects requiring muscle attachment (such as with eye balls), and the like.

The instant novel composites may be formed and tailored to have uniform and predetermined (or random) gradient orientations of bioactive fillers, particle size distributions, particle shapes, particle shape distributions, porosity, porosity distributions, and neat regions of pure polymer-matrix, depending on a desired target configuration. Bioactive particles with higher surface area and/or more edges dissolve faster and thus promote faster healing and regrowth while yielding the formation of porosity at a faster pace, which also tends to lower strength. Thus, the selection of the distribution of particle size, size distribution, shape, shape distribution, composition and surface area allows for the engineering of desired and predetermined growth and strength curves for the implant.

The bodies may be made porous prior to implantation by defining the pores with fugitive material that are removed prior to implantation, or the bodies may enjoy a porous interconnected architecture that emerges once implanted in vivo, as the bioactive spheres defining the pores are degraded in the body via a biologic response. The latter option typically yields a stronger implantable body.

Layered and/or gradient composites may be formed that would range from highly porous, highly bioactive at the outer surfaces to less porous, less bioactive in the core of the material. This flexibility is achieved via adjustment of the particle size, particle distribution, particle size distribution and shape of the bioactive particle additives, of fugitive additives and the parent polymer-matrix (PEEK) particles. In addition to PEEK, other biocompatible, load bearing polymer matrices (for example, PEEKEK and PLLA) are useful.

The radiopacity can be tailored from near radiolucent (nearly invisible under x-ray imaging) up to the radiodensity of human cancellous bone. Greater radiopacity is typically not desired, as assessing the growth of new bone throughout the implant body becomes more difficult if the radiopacity of the implant is too high (approaching, equal to or greater than the native cancellous bone). More typically the upper, lower and interior surfaces of the implant are more radiodense than the body of the implant 100, which allows for the location and orientation of the implant to be readily assessed radiographically.

Similarly, the mechanical properties are tailorable, relative to native cancellous and cortical bone. While the traditional solid and nonporous PEEK implants are nearly matched to the strength, toughness, and modulus of cortical cancellous geometries found in the human body, the instant implant incorporates bioactive glass fillers, directed porosity, and ultimate physiologic integration to make the time-zero properties starting points while the overall properties are dynamic as the physiologic bone formation and integration adds mechanical benefit to the implanted construct. Modulus, strength, and toughness are typically increased with the addition of bioactive fillers, which also typically increase with time post-implantation due to the incorporation of new bone tissue. Though emerging porosity can initially decrease the strength parameters, strength is still sufficiently high at time-zero to warrant augmenting human bone and will increase quickly and significantly as new bone apposition rapidly occurs, allowing for stress sharing at the bone-implant interface.

Porosity within these implants typically mimics human cancellous bone, in that the range of pore sizes is generally nanometer to millimeter, interconnected, rounded and all affording a greater surface area, per cross-sectional, axial area under load, that has exposed bioactive glass filler particles allowing for osteogenesis, angiogenesis and formation of nanocrystalline CaP bone precursors.

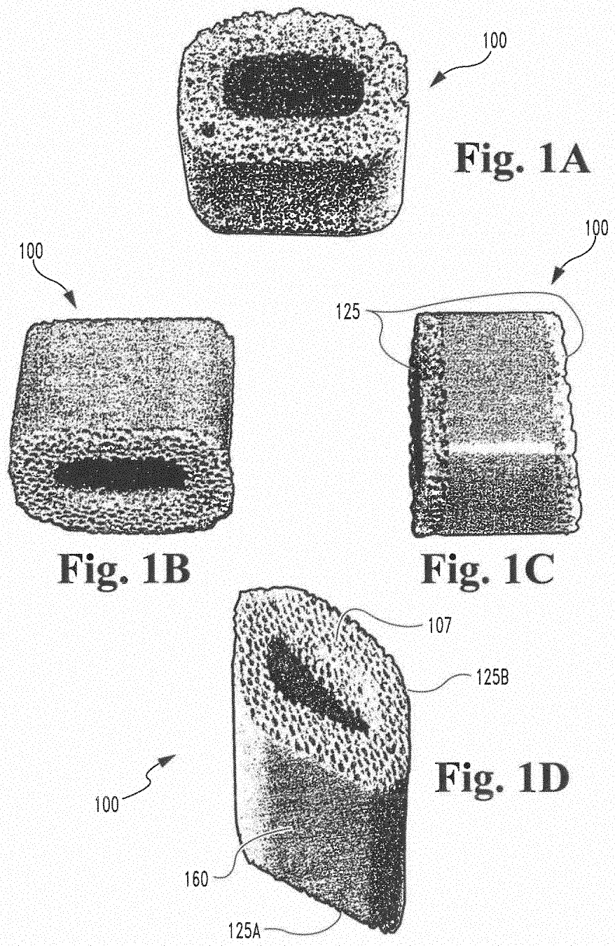

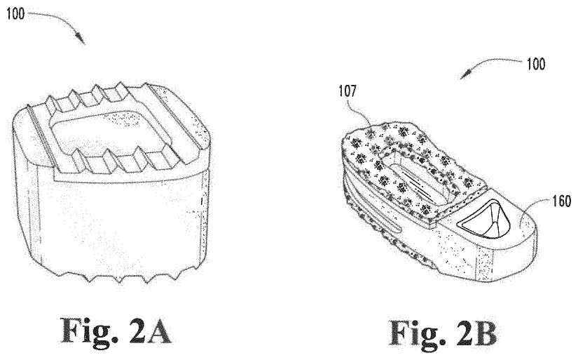

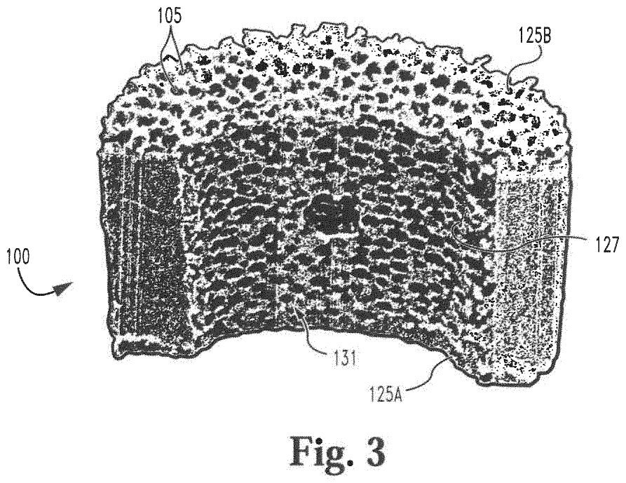

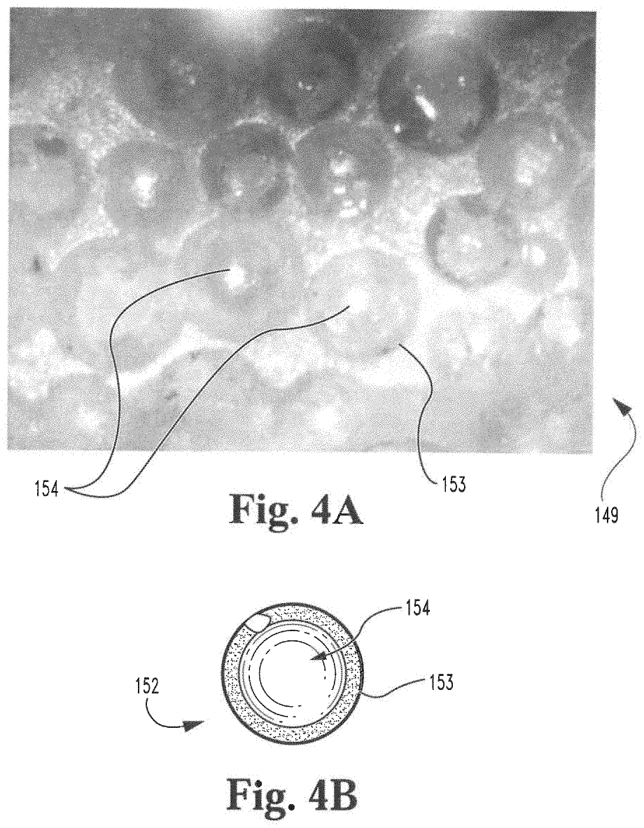

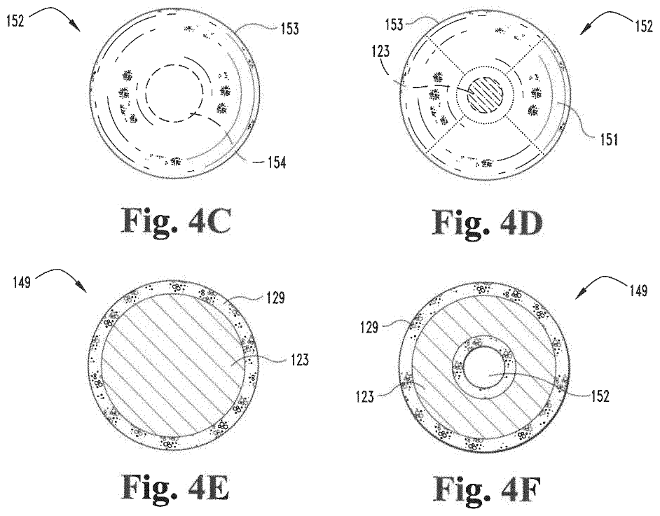

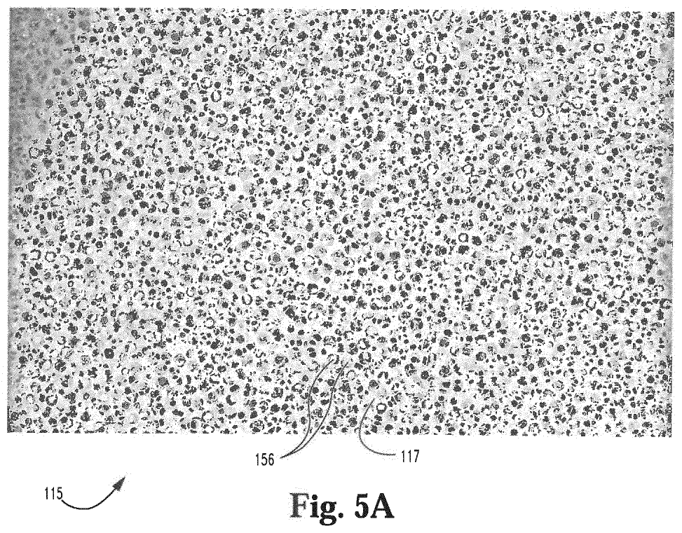

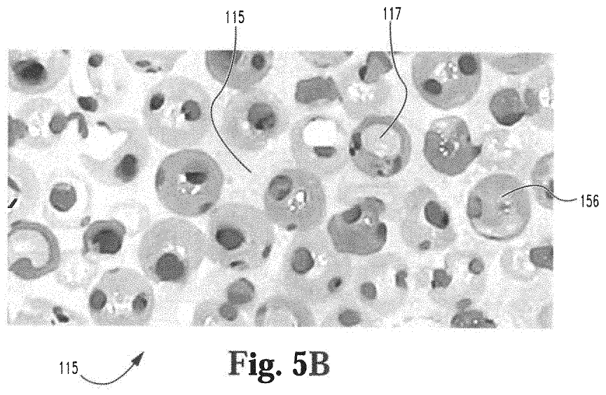

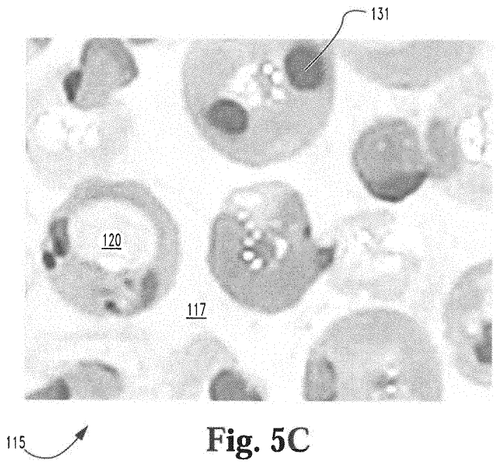

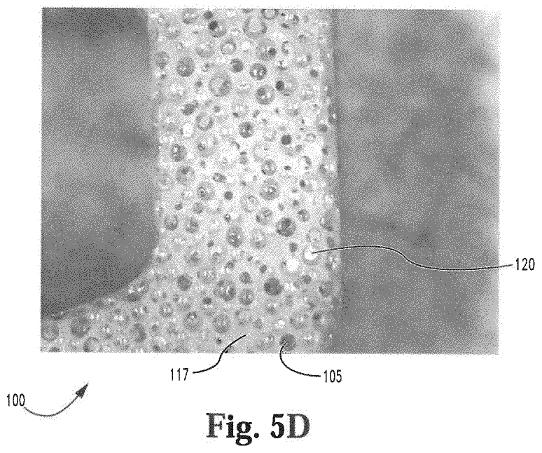

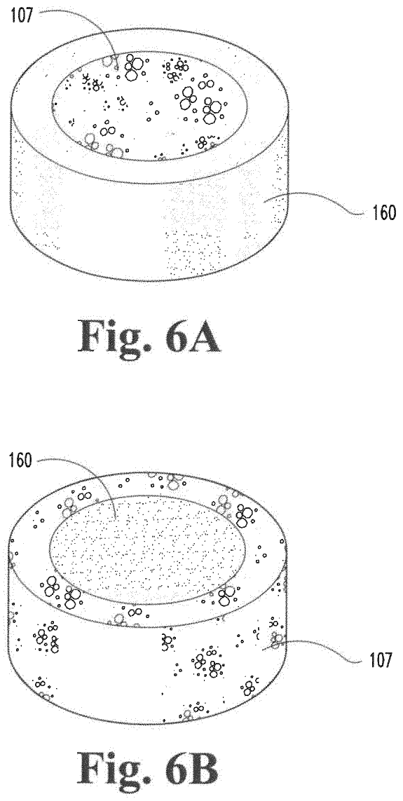

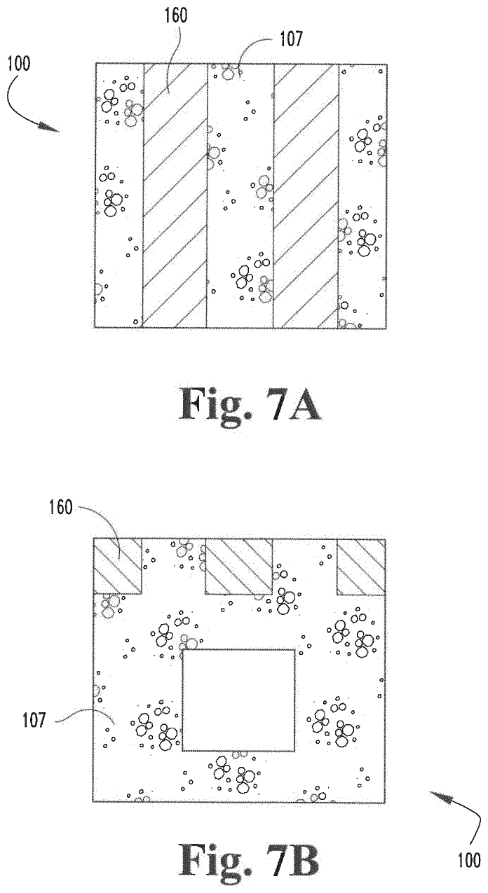

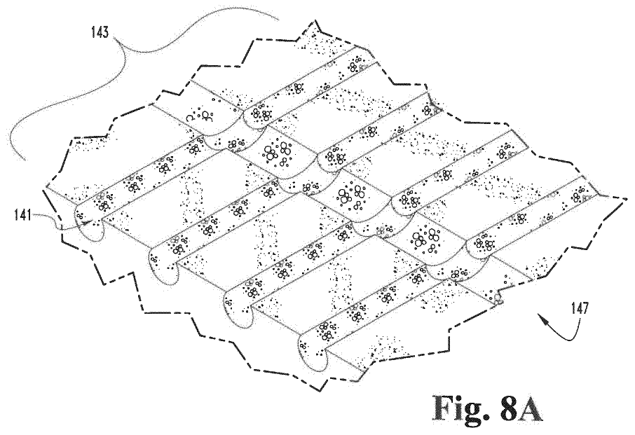

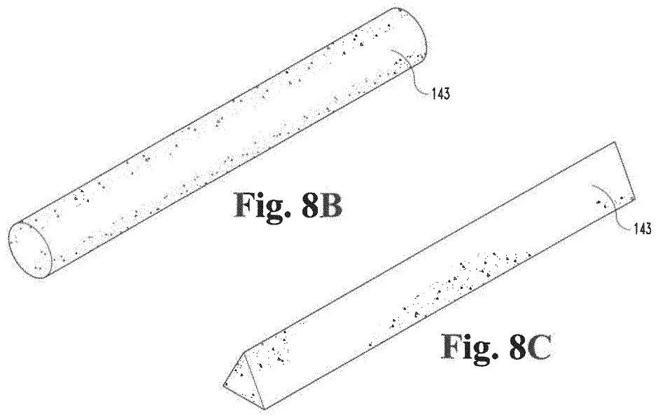

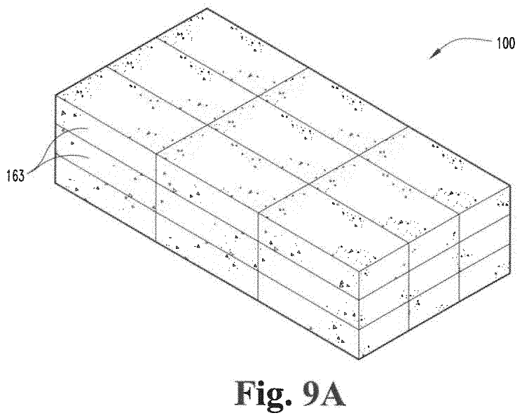

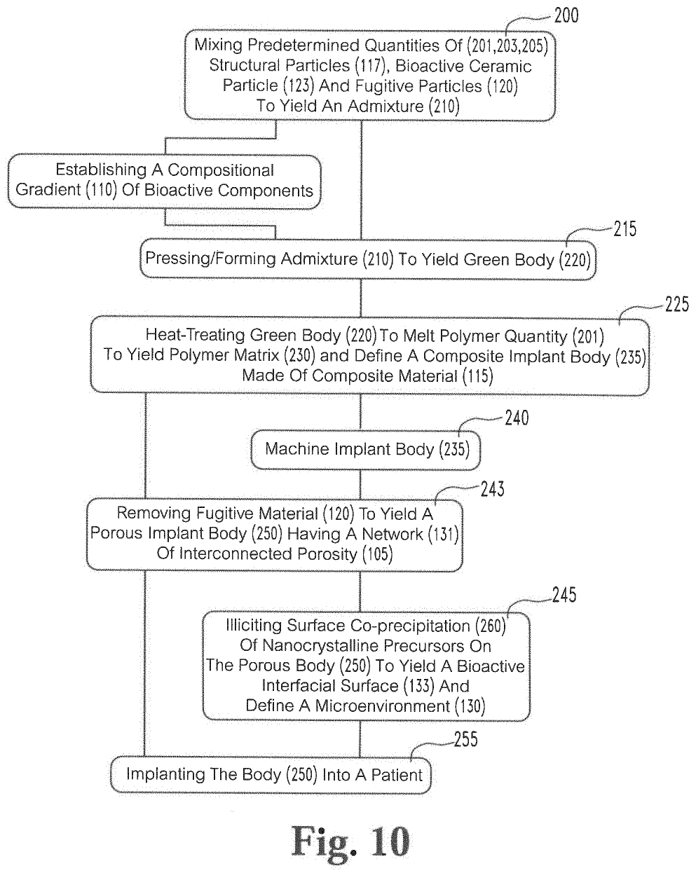

FIGS. 1-10 illustrate the present novel technology, implant devices 100 having radiopacity that may be made similar to that of bone or hard tissue and having variable and tailorable porosity 105 and bioactive compositional gradients no tailorable to a specific desired application and in vivo environment. The engineered and emerging porosity 105 is typically open and interconnected, and more typically greater near the outer bonding surfaces 103 of the implant body 100 to increase tissue (typically bone) bonding and tissue (typically bone) regrowth speed while reducing healing time and pain intensity and duration. The implant devices 100 are made of a composite material 115 that is typically a combination of a structural material matrix 117 (such as PEEK or like polymer material) throughout which a fugitive, and, typically bioactive, fugitive pore defining material 120 (such as bioglass) is distributed to define a pore network. The bioactive material is typically defined by a glass transition temperature (TG) and/or a melting temperature (TM) or softening temperature (TS), such as HA, btcp, PLLA, bio-glass, or like biologic response products, and is usually present in spherical, ovoid, or teardrop geometry. In other embodiments, the fugitive material 120 is a salt.

The composite material precursors 117, 120 are typically supplied as spheres or spheroids, but may also be present as particles, flakes, and/or fibers and/or combinations thereof. The composition, particle distribution, particle size distribution (PSD), particle shape, particle aspect ratio, porosity, and/or density may be varied to optimize healing time and pain relief and/or the strength and physical characteristics of the body 100, as well as to foster complete bridging of regrown tissue/bone, provide sufficient load bearing compressive strength, maximize tissue bonding, optimize connection time, and bond strength to hard and/or soft tissue, and to drive healing by promoting specific cell phenotypes.

Some differentiation features of the composite material 115 allow for accelerated healing, bone formation and apposition. This is typically achieved with accelerated reduction of micromotion, limited fibrous encapsulation of the implant 100, and preferred orientation evaluation due to the composite material 115 exhibiting sufficient radiopacity. The blending of bioactive and/or fugitive fillers 123, 120 at the interface surfaces 125 yields a predetermined porosity gradient, such as, for example, the outermost surface may be relatively rich in fugitive spheres and will thus yield greater porosity near the surface 125 than at the core 127. In other words, in this example the outer surfaces 125 will be more porous than the core 127, with more pores, larger pores, or both.

In another example, a body 100 may be formed having open porosity at the outer surface, such as by pressing the body 100 with fugitive material 120 (such as a salt) in the outer portions 125 and then removing the fugitive material 120 (such as with a solvent), and with a more solid core portion 127 containing an interconnected pore network 131 still filled with and defined by a bioreactive response material 123. The body 100 will have a greater core density and strength when inserted into a patient while exhibiting greater exterior surface porosity for promoting faster and more efficient integration of the body 100 with the recipient while limiting micro-motion to yield a pain-free patient more quickly. The bioactive interconnected architecture induces cells, tissue, and bone to build into the bioactive region of interconnected channels revealed as the bioactive material is released to provide nutrients driving growth.

The instant multicomponent composites 115 are useful for orthopedic, spinal and other general implant sites and benefit from more efficient, rapid and complete integration with, bonding to, and creation of bone apposition. The structure of the implant 100 is conducive to osteoconduction, typically having a porosity range between 100-500 microns. The creation of a bioactive interfacial surface 133 yielding an osteostimulatory and angiogenic microenvironment have been attained, at least in part, by the addition of bioactive ceramic, bio-glass, and/or bioactive glass-ceramic fillers 123, or like bioactive materials 123 (typically in spherical, ovoid, and/or teardrop geometry, although flake and/or fiber geometry may also be employed) and combinations thereof that yield mechanic strength, via composite principles, as well as modification of the implant surface 125 post implantation into the physiologic environment. Further, both the structural and the fugitive materials 117, 120 may be coated with specific bioactive coatings 129 to promote specific biological responses, such as at pore surfaces, implant interfaces, or the like. The surface reaction is thus a departure from the native or neat surface reaction typical of PEEK materials, widely used in orthopedic, spine implants.

The surface modification of the implants 100 benefit from the tailored integration of bioactive fillers 123. Known bioactive ceramics, such as bioglasses, have utility in this composition 115. One typical bioglass composition of this invention is the borate bioglass analogue designated 13-93B3, which is in weight percent given by

5.5 Na₂O-11.1 K₂O-4.6 MgO-18.5 CaO-3.7 P₂O₅-56.6 B₂O₃

which is known to be bioactive, osteostimulatory, and angiogenic. Another useful bioglass composition is, in weight percent, given by

24.5 Na₂O-24.5 CaO-6 P₂O₅-45 SiO₂.

Other ceramics, such as HA, beta-TCPs, monetite, brushite precursor CaPs may also be selected as appropriate additives by those skilled in the art. The selection of the particular bioactive glass composition, accompanied with various elemental additives (such as Sr, Cu, Zn, Mg, and the like) can further facilitate a microenvironment more conducive to bone formation and apposition, as well as promote the development and growth of particular, predetermined desired cellular phenotypes. The bioglass materials may be provided as powder precursors, derived from sol-gels, or any like convenient precursor process.

This surface reaction occurring at the tissue/implant interface 133 (known as bioactivity or osteostimulation) elicits Ca⁺² and PO₄⁻³ ion release into the microenvironment with the simultaneous change (to a negative) in surface charge. These surface reactions occur immediately upon implantation and may persist for weeks, or at least until the adjacent physiologic microenvironment interface has been converted to bone. The new bone-implant interface 133 will progress over time and transfer (transduce) mechanical loads through the interface 133, implant, and bone-implant system such as to avoid the formation of excessive fibrous tissue, encapsulation and resulting micromotion, implant loosening and possible failure. Additionally, the bioactive glass composition selected imparts angiogenesis or vessel formation, which also assists in the osteogenic process of bone formation, requiring blood, oxygen and cellular nutrients and protein precursors to infiltrate and support the surface reaction and microenvironment 130.

Also, the specific addition of bioactive glass particles 120 adds radiopacity to specific, upper, lower, and interior surfaces. This allows for the imaging, position and orientation of the implant 100. This obviates the need for added Ta or Ti metal pins or markers needed in traditional PEEK implants.

The desired reaction rate can be achieved with many different bioactive particles compositionally and size-wise. Ideally the particle size is sufficiently small as to not degrade significantly the mechanical properties of the resulting implant. Bioactive particles less than 100 microns across are typical, more typically less than 45 microns, and still more typically less than 10 microns and down to nanometer size range. Combinations of particle size modes (typically bimodal or trimodal, but may also be multimodal having four or more size modes) of larger and smaller particles may yield tailored emergent pore size distributions based on target levels of surface reactivity. Likewise, varied shapes of bioactive particle additives such as microspheres, irregularly shaped aggregates, fibers, rods, flakes, and combinations thereof all contribute to the efficacy of the implant 100 and may impart specific physical, chemical, biological and/or mechanical property benefits such as toughening, modulus adjustment and compressive strength. Coupling agents may or may not be employed or required, but can be exploited to increase the particle polymer-matrix interface strength.

Control of the rate and extent of implant 100 bonding to surrounding tissue may be related to the exposure of the implant interface 125 and is derived from the creation of actual or latent interconnected porosity 105 during manufacturing of the implant 100. The blending of a variety of component bioactive filler compositions, particle sizes, particle shapes, and/or PSDs yield various surface exposures of the incorporated bioactive glass as well as the generation of the interconnected porosity 105. One method used to generate controlled porosity 105 is achieved by the addition of non-bioactive fugitive materials 120, such as water soluble salts (typically NaCl) or other fugitive crystals, sugars, polysaccharides, combinations thereof, and the like, that can withstand the compression molding temperatures of the matrix polymer (PEEK) and subsequently be leached out of the implant 100 to yield voids and interconnected porosity 105 where desired in the final implant 100. The selection of the particle size, shape and distribution of the leachate fugitive component 120 is helpful in to controlling the microstructure and nature of the interconnected porosity 105 and resulting microenvironment 130 for bone formation. Round, spherical fugitive salt particles are typically selected in the size range of 100-600 micron fugitive particles yield larger cavities when dissolved away; the subsequent smaller fugitive particles 120 that reside in between the large particles 120, when leached out, allow for interconnectedness of the voids and, thus, interconnected open porosity 131.

By imparting fugitive material 120 into the body 100 in a specific, predetermined geometry, certain geometric features may be built into the body 100 that slowly manifest as the fugitive material 120 is removed, either by dissolution and/or in vivo. Geometric features 141 including storage volume shapes, such as channels and/or wells for storing, releasing, and delivering biomaterials, growth enhancers, drugs, or the like may be built into the body 100. Likewise, connection enhancement shapes 143 such as channels, voids, or dovetails for enhanced attachment of the implant body 100 to the host via tissue growth and ingrowth may be fashioned. The channels and like shapes 143 are typically impregnated with biologics 123 to engender a biologic response or release of agent as desired.

Creation of a dovetail or like semi-complex or complex geometrical feature 140, 143 may be accomplished by molding, 3-D printing, or like process without having to machine the implant body. The emplacement of fugitive material 120 allows development of three-dimensional structures 147 that may be surrounded and/or filled with a material 117 that survives removal of the fugitive material 120. For example, ten (10) mm fugitive spheres 149 may be overmolded by thirty (30) mm polymer balls 151, such that the fugitive portion 149 may be leached away to yield polymer shells 153 with internal void space 152 for filling with drugs, biologics, or ingrown tissue. The fugitive material 120 may have any convenient geometry (spherical, cubic, pyramidal, irregular, or the like), as can the exterior coating material 117.

Likewise, a fugitive body 149 may be formed over a core portion 154 (dissolvable or not) and the fugitive body 149 likewise encapsulated within an insoluble polymer body 151. Leaching away of the fugitive portion 149 leaves a soluble body core 154 within an insoluble body shell 151 for time release of the soluble body material 154, which may be a biologic or drug.

In some embodiments, a polymer matrix 151 is embedded with hollow salt spheres 149 to produce a structure 147 with porosity once the salt spheres 149 are leached away. The hollow void 152 within the respective salt spheres 147 may be filled with molten polymer (PEEK) 117 upon heat treatment so that there are elongated PEEK members 156 extending between interconnected pores 107 when the salt is leached out. The PEEK member 156 may be elongated tendrils, or may have teardrop, pear-shaped, or like droplet morphologies. Additionally, nanoscale bioactive particles and/or drugs 123 may fill the wells 152 within the hollow salt spheres 147, such as via vibration treatment, to yield an interconnected porous structure 115 with elongated PEEK members 156 and/or bioactives and/or drugs 123 molded into place.

The weight percent of added bioactive particles 123 is typically greater than 10%, more typically up to 20%, and still more typically between 20% and 60%. Increasing amounts of bioactive materials may lead to unacceptable decrease in strength, especially as the bioactive materials are dissolved. For applications requiring lower mechanical strength there is no practical limit, as long as at least 1-5% polymer-matrix persists for aggregate bonding.

Powder processing pre-molding impacts the distribution of the bioactive, polymer-matrix and fugitive phases 117, 120. The blend of multi-modally distributed particles 117, 120, 123, 149 will be compression molded at sufficient temperature, typically above 275 C and up to 350 C, to melt any polymer-matrix (PEEK) phase allowing for intimate incorporation and creation of the bulk composite 157. The bulk composite 157 can be formed and shaped by any convenient methodology, such as extrusion molded, near net-shape molded, cast or formed, machined or CNC formed into the near final implant shape 100. Any forming and/or machining is typically performed while the fugitive phase and/or bioactive phase material 120, 123, 149, 154 is still in place (not yet leached out) as to prevent any machining, forming debris from becoming entrapped in the inner interstices of the implant 100. After machining and/or forming, the implant 100 is submerged into warm water, saline, or other appropriate solvent to remove the soluble fugitive phase 120 material, leaving a typically interconnected network 131 of porosity 105 at the composite body surface 125 to yield a porous composite composition 115 typically comprised of PEEK polymer-matrix 117 infiltrated with bioactive spheres 123 positioned closer to the core 127 and defining the remaining as yet uncovered pore network 131 and that now have a significant exposure on the newly created implant interface 125 and an interconnected porous microenvironment 130.

Alternately, an implant body 100 may be formed by first preparing a structural material blend 115 wherein the outer portions 125 typically include structural material powder or microspheres 117 along with soluble fugitive powder or microspheres 120A, with the concentration of fugitive material 120A decreasing with distance from the surface 125, and a central or core portion 127 typically including a blend of structural material powder/microspheres 117 and bioactive powder or microspheres 120B. Of course, the outer portion 125 may include some amount of bioactive particles 123 and the core portion may include some amount of soluble fugitive particles 120. The body 110 is compression molded to yield a green body, and heated to TG but not TM. The remaining fugitive materials 120 may then be leached out with solvent to yield a body having an interconnected porous surface 125 with a more solid core 127 having filled pores that can be more slowly uncovered and filled with tissue growth.

Still alternately, bodies 100 may be engineered to have one or more solid portions that extend from the inferior surface 125A through the outer portion 125, through the core portion 127, back though the outer portion 125 and to the superior surface 125B, thus providing at least one solid load-bearing portion 160. The bodies 100 likewise have at least one porous portion 107 that likewise extend from the inferior surface 125A through the outer portion 125, through the core portion 127, back though the outer portion 125 and to the superior surface 125B, thus providing at least one porous portion 107 defining a body 100 made of a composite material 115 having an interconnected porous structure 131. The at least one porous portion 107 may include actual pores 105 and/or fugitive materials 120 defining potential emergent porosity.

Powder blending relates to the addition of appropriate amounts of the respective filler particles (if any), structural polymer powders, pore defining bioactive ceramic powders and pore defining fugitive powders. Typical blending and/or powder homogenization methods can be performed dry or with solvent assistance. When using solvents, residual solvents are typically removed prior to compression molding. Dispersion of the respective particles can be achieved using basic agitation, blending, shaker methods known well in powder processing.

In some embodiments, pre-conditioning or pre-reacting of the bioactive particles 123 is done to expose the surfaces of the implants 100 by soaking final implants 100 in PBS (Phosphate buffered Saline), SBF (simulated body fluids), or like solutions for appropriate periods of time to illicit the co-precipitation of nanocrystalline (CaP) HA precursors, CaPs on the surface of the composite prior to implantation. This can be done during manufacturing, after the salt leaching step, pre-implantation and could accelerate protein, cell, or drug attachment once implanted.

In some embodiments, the implant body 100 has substantially solid or nonporous outer surfaces 160 or ends with a substantially porous network portion 131 extending therebetween.

The pore structure 107 of the implant body 100 may be controlled through the selection structural material 117 and fugitive material 120, 149 particle shape, particle size, PSD, and modality, and relative distribution of structural and fugitive particles. For example, by selecting all spherical particles of a single modality (or size), maximum packing density of undeformed spheres is about seventy-four percent, yielding a twenty-six percent porous body having homogeneously distributed pores or voids. Packing density may be increased by selecting a multimodal (two, three, or more different sphere sizes) to fill voids with smaller spheres.

Pores may be non-homogeneously distributed by directed layering of the structural 117 and/or fugitive materials 120, 149 (such as by 3D printing or the like), intentionally leaving voids to yield a predetermined pore structure 107. Likewise, a nonhomogeneous distribution of fugitive materials 120, 149 may allow for the generation of pores 107 upon exposure to solvents, heat, or other conditions. Similarly, bioactive materials 123 may be non-homogeneously distributed in the body 100 to yield emergent pores 107 once the implant 100 is placed in vivo. Blends of particle sizes, shapes, and compositions, along with variations in distribution within the body 100 may be combined to yield a body 100 having specifically tailored initial porosity, pore distribution, and PSD as well as a predetermined pore evolution and/or directed ingrowth of tissue not the body 100 once the body 100 is implanted in vivo. Thus, the above-described geometric and compositional factors may be used to control and predetermine such elements as the penetration, location of tissue ingrowth, and the orientation and/or direction of tissue ingrowth.

Adjustments to the orientation of the various particles 117, 120, 123, 149, 154 combined in the pre-molding step can achieve gradient compositions that have inner cores 127 of pure PEEK, interior surfaces of that inner core 127 having higher percentages of the bioactive particles 123, thus allowing for multiple resulting surfaces having bioactivity while retaining the innate biomechanics of the polymer-matrix 117 (typically PEEK). In particular, an interbody spine implant (IBFD) 100 would be able to achieve bioactive interfaces 133 in a composites superior, inferior and central-interior surfaces. The superior and inferior interfaces of the spine implant would be in direct contact with the bone of the adjacent vertebrae being “fused” and would exist in compression loading, which would facilitate the rapid formation of bone.

Additional methods of composite formation may be accomplished using the precursor powder blends described above, such as 3-D printing of layers using known methods. This would increase the ‘tuneability’ of the various layers, interfaces, surfaces and the achievement of precision, complex shapes and configurations.

Complex shapes, porosities and surface bioactivity may be achieved by using combinations of material, additives, fugitives, as well as shapes, sizes and PSDs of the particles, to yield implants 100 having superior surfaces and bulk characteristics for cell attachment, protein adsorption and drug or antibiotic incorporation at the time of implantation or during manufacture. Low-temperature post fugitive leachate and infusion allows for incorporation of temperature sensitive drugs, molecules, proteins, and the like.

Increased porosity 105 can reduce the original strength expected from a neat implant (no pores), but even with significant porosity 105, such as up to 70% or even 80% or more, the implant body 100 can still retain sufficient strength, axial compression strength, shear strength, and elastic modulus acceptable for physiologic loading in the human skeleton, and, in particular, the spine. The incorporation of bioactive fillers 129 can increase the elastic modulus of the resultant body 100 and by promotion of bioactivity, bone apposition at the interface 125 can lead to an increase in the overall strength and load capacity of the implant-bone system. Thus, increased porosity 105 increases the surface area exposed to the physiologic microenvironment 130 that promote bone formation, apposition, angiogenesis and ultimately implant-bone stability. The instant implants 100 have increased surface area for bioactivity per axial area in between bone segments to be fused and loaded.

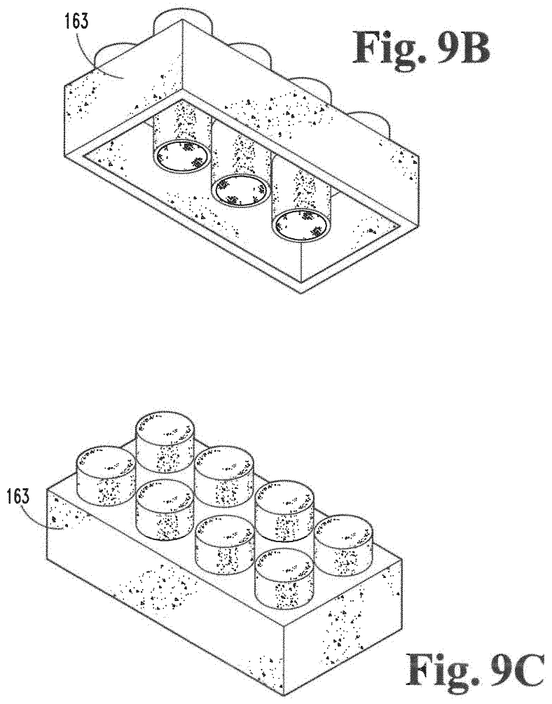

As illustrated in FIGS. 9A-9B some implant bodies 100 are formed as versatile, expandable technology, wherein a solid, fugitive material interior portion 127 is encapsulated within one or more porous outer portions 125 that engage the solid inner portion 127 with an interference or snap-on fit (much like LEGO® toys; LEGO is a registered trademark of LEGO JURIS A/S CORPORATION, DENMARK, KOLDINGVEJ 2 BILLUND DENMARK DK-7190, registration no. 1026871, Dec. 9, 1975).

Smaller units 100 may be engaged with one another to yield a large unit having a specifically tailored size, shape, and composition. Even spherical bodies having outer and inner portions 125, 127 of different porosity and/or composition may be produced in this way. In one example, a porous shell 125 of either inert or bioactive material surrounds a bioactive agent core 127; in another example, a biomaterials shell 125 surrounds an outer bioactive core 117 surrounding a hollow inner core 128 made of a different bioactive material to yield a time-released bimodal bioactive delivery vehicle.

In some examples, a porous, bioactive implant 100 is partially encapsulated or coated with a non-tissue adhesive polymer film 161 to further predetermine where tissue adhesion and ingrowth occurs/does not occur. In still other embodiments, implants 100 may be made with discrete porous 107 and solid portions 160 to govern where adhesion and ingrowth occur to provide load-bearing structural support to the body 100, and/or to provide a solid or substantially less porous leading edge to protect the porous surface(s) of the implant body 100.

In some embodiments, the implant body 100 includes an embedded lattice structure 163 for supporting materials of different shapes and compositions to further direct and influence tissue ingrowth. For example, the lattice may be similar to a tongue and groove structure, wherein elongated rectangular or cylindrical members or a different composition and/or porosity are accepted therein to provide porous ingrowth avenues or internal structural support.

In operation, the implant 100 is produced by first mixing 200 a first quantity 201 of structural, typically bioactive, polymer particles 117, a second quantity 203 of bioactive ceramic particles 120B, and a third quantity 205 of fugitive material particles 120A to define an admixture 210. The relative amounts of each portion 201, 203, 205, as well as the sizes, PSDs, shapes, shape distributions, and compositions thereof, are selected to cooperate to yield a predetermined degree and distribution of porosity, a predetermined target dissolution rate of the bioactive portions, and a predetermined target strength of the implant 100. The admixture 210 is pressed or otherwise formed 215 to define a green or composite body 220, which is then typically heat-treated 225 or calcined to melt the first quantity 201 of bioactive polymer particles 117, defining a calcined composite implant body 235 having bioactive ceramic particles 120B and fugitive material particles 120A bound in a bioactive polymer matrix 230. The calcined body 235 may then be machined or otherwise formed 240 to a desired shape, if necessary. The calcined body 235 may then be infiltrated 243 with a solvent to remove 245 the some or all of the fugitive material particles 120A to yield a network of interconnected pores 105 and to define a porous calcined body 250 ready for implanting 255 into a patient 265. The network of interconnected pores 105 may be thought of as being preexisting and filled with the fugitive material particles and/or the bioactive particles. Optionally, prior to implantation 255, the surface co-precipitation 260 of nanocrystalline (CaP) HA precursors and/or an infusion of other biologically active materials (drugs, proteins, or the like) into the porous calcined body 250 may be elicited.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

We claim:

1. A method of manufacturing an implant, comprising:
a) mixing a first quantity of biocompatible polymer particles, a second quantity of bioactive ceramic particles, and a third quantity of fugitive material particles to define an admixture;
b) forming the admixture to define a composite body having an inferior portion, a superior portion and a central portion disposed between the inferior and superior portions;
c) heating the admixture to fuse the first quantity of bioactive polymer particles to define a composite implant body; and
d) infiltrating the composite implant body with a solvent to remove fugitive material particles to yield a network of interconnected pores and to define a porous implant body;
wherein the fugitive material particles are hollow spheres partially filled with a material selected from the group comprising air, bioactive agents, biological growth enhancers, drugs, and biocompatible polymer material, combinations thereof.

2. The method of claim 1 wherein the composite body is formed by three dimensional printing; and wherein the superior and inferior portions are more porous than the central portion.

3. The method of claim 1 wherein during step c, the hollow fugitive material spheres are partially filled with biocompatible polymer and after step d, elongated members of biocompatible material extend between respective interconnected pores.

4. The method of claim 1 wherein during step b, the hollow fugitive material spheres are partially filled with biocompatible polymer and after step d, elongated members of biocompatible material extend between respective interconnected pores.

5. The method of claim 1 and further comprising:
e) before step d, forming predetermined geometric features into the body with fugitive materials such that the geometric features emerge during step d.

6. The method of claim 5 wherein the geometric features are selected from the group comprising connection enhancement shapes, storage volume shapes, and combinations thereof.

7. The method of claim 1 wherein the respective particles define a multimodal size distribution; and wherein the particles define at least four different size modalities.

8. The method of claim 3, wherein the elongated members are teardrop shaped.

9. The method of claim 1 and further comprising:
f) implanting the porous implant in a patient.

10. An implant body, comprising:
a body volume having an inferior portion, a superior portion, and a central portion disposed between the inferior and superior portions;
a first biocompatible polymer portion distributed throughout body volume;
a second bioactive portion distributed throughout the body volume; and
a third interconnected fugitive material portion distributed throughout the body volume and defining potential interconnected pores to be realized upon removal of the fugitive material portion;
wherein the bioactive portion is encapsulated in a material selected from the group comprising biocompatible polymer, fugitive material, an adhesive, and combinations thereof;
wherein the central portion is less potentially porous than the inferior and superior portions; and
wherein the potential pores in the inferior portion are on average larger than the potential pores in the central portion.

11. The implant body of claim 10 wherein at least some of the fugitive material portion defines a shape selected from the group comprising wells, channels, and dovetails.

12. The implant body of claim 10 wherein at least some of the fugitive material portion defines a shape selected from the group comprising spheres, cubes, and pyramids.

13. The implant body of claim 10 wherein the fugitive material portion further comprises a plurality of hollow salt spheres.

14. The implant body of claim 13 wherein the respective hollow salt spheres contain biocompatible polymer.

15. The implant body of claim 13 wherein the respective hollow salt spheres contain bioactive agents.

16. The implant body of claim 10 wherein the second bioactive portion is a plurality of bioactive particles, and wherein each respective bioactive particle is encapsulated in a material selected from the group comprising fugitive material, biocompatible polymer, and combinations thereof.

17. The implant body of claim 10 wherein the respective portions define a multimodal particle size distribution.

18. The implant body of claim 17 wherein the multimodal size distribution includes at least four different size modalities.

19. The implant body of claim 10 wherein at least one solid portion extends from the inferior portion through the central portion and through the superior portion; and wherein at least one porous portion extends from the inferior portion through the central portion and through the superior portion.

20. The implant body of claim 10 and further comprising at least one recess formed therein and at least one connection member extending therefrom; wherein the at least one recess is sized and shaped to accept the at least one connection member to define an interference fit.
